(19) United States
(12) Patent Application Publication
(10) Pub. No.: US 2009/0247498 A1
(43) Pub. Date: Oct. 1, 2009

(54) Pharmaceutical Compositions Primarily for the Treatment and Prevention of Genitourinary Infections and their Extragenital Complications

(76) Inventor: Milankovits Marton, Budapest (HU)

Correspondence Address: HAUGEN LAW FIRM, SUITE 1130 - TCF TOWER, 121 SOUTH EIGHTH STREET, MINNEAPOLIS, MN, 55402, US

(21) Appl. No.: 12/482,090

(22) Filed: Jun. 10, 2009

Related U.S. Application Data
(63) Continuation-in-part of application No. 12/353,320, filed on Jan. 14, 2009.
Application No. 12/353,320 is a continuation-in-part of application No. 12/274,535, filed on Nov. 20, 2008.
Application No. 12/274,535 is a continuation-in-part of application No. 11/076,194, filed on Mar. 9, 2005, now abandoned.
Application No. 11/076,194 is a continuation-in-part of application No. 10/192,345, filed on Jul. 10, 2002, now abandoned.
Application No. 10/192,345 is a continuation-in-part of application No. 08/776,273, filed on Jan. 22, 1997, now Pat. No. 6,432,935.

(51) Int. Cl. A61K 31/431; A61P 25/00

(52) U.S. Cl. 514/196

(57) ABSTRACT

Compositions having an effective amount of an antibacterial agent may be useful in the treatment of amyotrophic lateral sclerosis (ALS) and other diseases of the nervous system. The compositions may include an extraordinary amount of an antibacterial agent. Ceftriaxone has stopped the progression of ALS for a second time after nearly 18 years of the disease.

### CROSS-REFERENCE TO RELATED APPLICATION

This application is a continuation-in-part of U.S. application Ser. No. 12/353,320, filed on Jan. 14, 2009 and entitled “Pharmaceutical Compositions Primarily for the Treatment and Prevention of Genitourinary Infections and Their Extragenital Complications”, which itself is a continuation-in-part of U.S. application Ser. No. 12/274,535 filed Nov. 20, 2008 and entitled “Pharmaceutical Compositions Primarily for the Treatment and Prevention of Genitourinary Infections and Their Extragenital Complications”, which is a continuation-in-part of U.S. application Ser. No. 11/076,194, filed Mar. 9, 2005 and entitled “Pharmaceutical Compositions Primarily for the Treatment and Prevention of Genitourinary Infections and Their Extragenital Complications”, which is a continuation-in-part of U.S. application Ser. No. 10/192,345, filed Jul. 10, 2002, and entitled “PHARMACEUTICAL COMPOSITIONS PRIMARILY FOR THE TREATMENT OF GENITO-URINARY INFECTIONS”, which application itself is a continuation-in-part of U.S. application Ser. No. 08/776,273, filed Jan. 22, 1997, and entitled “VAGINAL SUPPOSITORIES AND NOVEL PHARMACEUTICAL COMPOSITIONS”, the contents of which are herein incorporated in their entirety.

### FIELD OF THE INVENTION

This invention relates to novel compositions and their uses in treatments of amyotrophic lateral sclerosis (ALS) and other diseases of the nervous system.

### BACKGROUND OF THE INVENTION

A variety of vaginal suppositories are currently commercially available for the treatment of various maladies. The attending physician ordinarily decides which composition is best suited to the patient's needs following physical examination.

For example, for the treatment of vaginal mycosis, Canesten (active ingredient is clotrimazol; bis-phenyl-(2-chlorophenyl)-1-(imidazolyl)-methane; and Pimafucin (the active ingredient is natamycin-primaricin) are most commonly used. For fungal and protozoan infection, Klion D (the active ingredient is metronidazole; 1-(2′-hydroxiethyl)-2-methyl-5-nitroimidazol and myconasol-nitrate) is used. For protozoan infection, Klion vaginal suppository (active ingredient is metronidazole) is commonly used.

Certain compositions exert their effects through the disinfective action of iodine. These include Betadine (iodine is released from the carrier). Other vaginal suppositories feed the natural flora of the vagina. These include Genia 92 nutrients, e.g.: folic acid, lactic acid, lactose, and lactamine.

A common disadvantage of the above compositions is that none of them makes possible the combination of the effects of (i) bactericide (for aerobe and anaerobe bacteria), involving anti-Mobiluncus and anti-Gardnerella, (ii) fungicide, and (iii) anti protozoa simultaneously. Moreover, they have no antiviral effect at all.

### SUMMARY OF THE INVENTION

The invention relates to compositions which make possible the attack of pathogens from different directions, which simultaneously aid in the body's antiviral struggle. The invention facilitates rapid and simple selection of the safest and most useful compositions.

The basis of the invention is the discovery that a unique combination of active ingredients has numerous advantages over the art. In a preferred embodiment, the composition of the present invention includes an antibacterial agent, an antifungal agent effective against a Candida species, and a nitroimidazole, wherein the antibacterial agent, the antifungal agent, and the nitroimidazole are present in the composition in synergistic effective amounts. Preferably, the compositions of the present invention further include a pharmaceutically acceptable carrier.

The basis of the discovery according to the invention is that the effect of the antibacterial agent ingredient is unexpectedly intensified by the other active components of the present composition. For example, the antibacterial effect, particularly of chloramphenicol and sulfonamide, against Chlamydia trachomatis is greatly increased by the present compositions. Additionally, an increased inhibitory effect of the antibacterial agent, particularly chloramphenicol and nitroimidazole, particularly metronidazole, components against anaerobe pathogens (e.g. B. fragilis) was observed when utilized in compositions of the present invention. The antibacterial effect of the sulfonamide and/or nitroimidazole also unexpectedly potentiated antibiotics generally against each pathogenic bacterium, in the antifungal protection provided by the antifungal agent. The antifungal agent or agents play a minor role in the synergistic effect.

The unexpected increased effectiveness of the components of the present composition when present in synergistic effective amounts has been observed when utilizing the three ingredients listed above. A further aspect and embodiment of the present invention provides for the inclusion of a sulfonamide in addition to another antibacterial agent, the antifungal agent, and the nitroimidazole, when present in a composition in synergistic effective amounts. As such, another embodiment of the present invention comprises an antibacterial agent, a sulfonamide, an antifungal agent effective against a Candida species, and a nitroimidazole, wherein each of these components are present in a composition in synergistic effective amounts. Preferably, the composition further includes a pharmaceutically acceptable carrier for obtaining a suitably deliverable medication to a patient.

It has been observed through experimental data that the antibacterial effect of sulfonamide standing alone is greatly enhanced when coupled with the other ingredients of the present compositions. Therefore, such an unexpected increased effectiveness of sulfonamide when incorporated into the compositions of the present invention forms an additional embodiment and aspect of the present invention.

An additional aspect and embodiment of the present invention further supports the unexpected increased effectiveness of sulfonamide when combined with other active ingredients. A particular example which has proven unexpectedly effective against bacterial vaginosis is the combination of a sulfonamide and a nitroimidazole.

Thus, the treatment spectrum is broader and the effect of the combination is much stronger than would be expected from its individual components, while simultaneously decreasing the necessary dosage for treatment compared to the individual active ingredients. This results in a decrease of the possible side effects while using the present combination of elements. Another significant advantage of the solution according to the invention is that drug resistance does not occur. The main role in the synergistic effect is played in the compositions of the invention by the nitroimidazole and/or sulfonamide component(s), probably causing apoptosis of the involved eukaryotic cells and by killing most of the prokaryotic cells. (Apoptosis-like additional effect). The level of the mechanism of action is the same in the case of a nitroimidazole or a sulfonamide. Both of them act on transcriptional level. The unity of the invention more explicitly is the following: The basis of the invention is the discovery that the nitroimidazoles and sulfonamides potentiate the effectiveness of each other and/or the effectiveness of other antimicrobial agents. Both the nitroimidazoles and the sulfonamides impair the intracellular metabolism and act on DNA level too. Each of them weakens the target cells (The target cells are prokaryotic cells and unhealthy eukaryotic cells). Thus, the second antimicrobial agent or agents of the compositions of the invention is/are unexpectedly effective on the weakened target cells (the sulfonamide component can be replaced by a combination of a sulfonamide and trimethoprim, because their combined effectiveness on cell metabolism is well known).

An additional aspect and embodiment of the present invention is the group of new molecules containing the radicals of the components mentioned above. The antifungal component(s) effective against Candida of the present invention further contribute to the unexpected increased efficacy of the compositions. Problematic super-infections with Candida occur very frequently and this is the second important reason why necessary combined antifungal treatment and the antifungal components should be effective against Candida. Similarly to the combined antibacterial treatment, the combined antifungal treatment is much more effective than the monotherapy. The compositions of the present invention are equally effective in the cases of systemic and local treatments and shorten the duration of the antigen stimulus which may result in the need of vaccination. A treatment, however, without a full “sterilizing” dose of a suitable substance results in the development of “serum resistant strains” (Paul Ehrlich, Partial cell functions, Nobel Lecture, Dec. 11, 1908).

### DETAILED DESCRIPTION OF THE INVENTION

The present invention relates to novel compositions having synergistic effective amounts of one or more antibacterial agents, a nitroimidazole, and an antifungal agent effective against a Candida species. The antibacterial agents used in the present invention are also commonly referred to as antibiotics. The present invention contemplates the use of any antibiotic as defined by Martindale—The Extra Pharmacopoeia, 29th Ed, London, The Pharmaceutical Press, 1989 (hereinafter “MARTINDALE”). As stated in MARTINDALE, page 94:
Antibiotics have traditionally been divided into bacteriostatic antibiotics which reversibly inhibit the growth of susceptible microorganisms and bactericidal antibiotics which kill the organisms in vitro. Given in high therapeutic doses, the aminoglycosides, cephalosporins, penicillins, and polymyxins are generally bactericidal by this criterion whereas chloramphenicol, erythromycin, the sulfonamides, and the tetracyclines are usually bacteriostatic. However, an antibiotic which is bactericidal in a certain concentration may become bacteriostatic at lower concentrations.

In addition, antibiotics are classified into five classes based on chemical structure and mechanism of action in Chapter 43 of Goodman & Gilman's The Pharmacological Basis of Therapeutics, 9th Ed. (McGraw-Hill, 1996) (hereinafter “GOODMAN-GILMAN”). Such classes are set forth in Chapter 43, pages 1029-1030 of GOODMAN-GILMAN. As described therein, the individual members of each class are interchangeable with one another since they act on the target microorganism in the same way, and in most cases are also chemically interrelated with one another.

Experimental data set forth in the examples detailed below indicate that antibiotics belonging to each of the five classes defined in Chapters 43-48 of GOODMAN-GILMAN are effective and preferred for use in compositions of the present invention.

In some embodiments of the present invention, it is desired to utilize a sulfonamide for the particularly desirable antibiotic effect thereof when used in combination with the other components of the composition of the present invention. Any sulfonamide may be utilized in the composition of the present invention as all sulfonamides are structural analogs and competitive antagonists of para-aminobenzoic acid (see GOODMAN-GILMAN page 1058). Though all sulfonamides are interchangeable as applied to the composition of the present invention, a particularly preferred sulfonamide is sulfadimidin. In applications where a patient has an allergy to sulfonamides, it is preferred to formulate the composition of the present invention without inclusion of a sulfonamide.

The antifungal agents utilized in the compositions of the present invention are preferably effective against Candida species (Candida albicans, Candida stelloidea, etc). Antifungal agents are divided into groups in GOODMAN-GILMAN, Chapter 49, pages 1175-1190, defining antifungal agents effective against Candida species which act only topically, and those agents effective against Candida species both systemically and topically. The antifungal agents effective against Candida species which act both systemically and topically are divided into the following subclasses: polyenes, azoles, and pyrimidines. According to GOODMAN-GILMAN, Chapter 49, members of each subclass are interchangeable, in that each member of a respective subclass exhibits similar chemical properties and functionalities. Characteristic representatives of the polyenes are natamycin and nystatin. A characteristic representative of the azole subclass is clotrimazole. A characteristic representative of the pyrimidine subclass is flucitozine.

Another subclass of antifungal agents effective against Candida species are those antifungal agents which act only topically. Characteristic representatives of this subclass are ciclopirox olamine, naftifine, terbinafine, and haloprogin. Though the antifungal agents selected for use in the compositions of the present invention are preferably effective against Candida species, it is not necessary that such antifungal agents be effective solely against such Candida species. For example, particular selected antifungal agents of the present invention may be effective against a multiplicity of fungi.

The nitroimidazole component of the compositions of the present invention further contributes to the unexpected increased efficacy of each component of the compositions. The mechanism of action of the nitroimidazoles is set forth in GOODMAN-GILMAN, page 996 as reflecting a selective toxicity to anaerobic or microaerophilic microorganisms. As such, the members of the nitroimidazole group share a common functionality and are so closely related in structure so as to be interchangeable (see GOODMAN-GILMAN). Characteristic representatives of the nitroimidazole group are metronidazole and tinidazole.

In one embodiment, the composition of the present invention includes:

| Component | Amount |
|---|---|
| a) Antibacterial Agent | 0.04-0.30 g |
| b) Antifungal Agent | 0.025-0.30 g |
| c) Nitroimidazole | 0.10-0.45 g |

A pharmaceutically acceptable carrier for the compositions of the present invention is polyethylene-glycol, but other suitable carriers may also be employed. The amount of polyaethylene-glycol supplements the combination of the active ingredients to the necessary amount in case of a 10 unit package.

One aspect of the present invention is the unexpected increased efficacy of the presently disclosed components when utilized in the compositions of the present invention, as contrasted with the minimal effectiveness of such components standing alone. Commercially available medications containing only one component of the compositions of the present invention have been found to be ineffective, even after extended treatment periods or administration of large doses. Monotherapy, which is the treatment with a single active ingredient, is ineffective, in that a relapse rate of vaginal bacterial vaginosis is about 30-35% of the patients receiving such monotherapy, even over a treatment duration of up to 20 days.

Through clinical trials, Applicant has discovered an unexpected synergy of effectiveness in compositions consisting of the three active ingredients referred to above.

The combination of elements used in the compositions of the present invention is more effective and qualitatively different from the separate administration of the individual components. Such compositions may be contained and administered in the form of vaginal suppositories, ointments, vaginal drops, talc powders, and painting solutions. Administration of the present compositions has resulted in complete recovery in cases when recovery could not be reached by the separate administration of the components. The compositions are useful in a variety of applications and treatments, including the following:

Prophylactic Use. Use of the present compositions prevent infection from infected swimming-pool water or sexual activity. The compositions are indispensable prior to gynecological operations (especially utero-vaginal interventions) as a prophylactic suppository.

Treatment of Infection. For infections, use of a preparation containing a composition of the present invention results in absolute recovery in 90% of the cases. Since systemic treatment is not needed, a smaller dose is administered. Cessation of treatment results in side effects disappearing (for local treatments there were no side effects observed). Resistance of the pathogens against the components used is also obviated because in local treatment the relatively small amount of preparation applied absolutely kills the pathogens.

Treatment of Chronic Vaginitis and the Alterations of the Cervix and of the Uterus. Presently, techniques such as laser surgery, conventional surgery, cryocoagulation or electro-cauterization are used for the treatment of the positive epithelial differences such as the acetic acid positive epithelium and P3 or repeatedly P3 cytological findings.

The compositions of the present invention promote the spontaneous healing of the bleeding, inflamed portion of the uterus. As a result, the above-described invasive conventional treatments become unnecessary. Constant inflammation plays a decisive role in the formation of cancer of the cervix of the uterus. Application of the compositions according to the invention greatly diminishes the risk of the formation of the cancer of the cervix of the uterus, by stopping the inflammation. Nitroimidazole has certain anticancer effects, which are observed during the radiation treatment of the tumors, where it increases the efficacy of the radiation treatment. In addition, it has been postulated that sulfonamides display anti-tumor characteristics as well (see Supuran C T et al. Carbonic Anhidrase Inhibitors: Sulfonamides as Antitumor Agents? Biorg. Med. Chem., March, 2001, 9(13):703-714).

Following the treatment with the present compositions, the laboratory findings improved from P3 cytological results to P2 or P1 without exception and the epithelium became normal kolposcopically.

Treatment of Complications of Ascending Infections. Ascending infections such as Cystitis, PID, etc. are typically caused by the same microbes as are in the vagina. The systemic application of the present combinations unexpectedly shortens the usual duration of treatment.

Later complications of the inflammation (infection) can be vascular damage anywhere in the body (e.g. atherosclerosis and the connected diseases (see Ross, R. Mechanism of disease: Atherosclerosis—an inflammatory disease. N. Engl. J. Med. 1999, 2:115-126), neovascularisation, etc.).

Chronic inflammation has been shown to be an important risk factor for a variety of epithelial cancers, including those of the esophagus, stomach, pancreas, liver, biliary tract, colon, vulva, and bladder (see Stephen E. Hawes, Nancy B. Kiviat. (editorials) Are Genital Infections and Inflammation Cofactors in the Pathogenesis of Invasive Cervical Cancer?, Journal of the National Cancer Institute 2002, 94:1592-1593).

Antiviral Effect. The present combination acts against viral infections, possibly by killing all non viral pathogens, enhancing the immunocapacity of the body against the viruses.

In those cases when, because of inflammation and vaginal discharge, the treatment of the Condyloma acuminatum (caused by the human papilloma virus) failed, additional local treatment using the present composition proved to be permanently successful.

Moreover, it was observed that patients with frequently reoccurring herpes genitalis, became permanently free of symptoms following the treatment with the preparation of the present invention.

The preparation according to the invention can also be administered to pregnant women to prevent adverse pregnancy outcome (and probably toxemias of pregnancy too). In such applications, erythromycin is the preferred antibacterial agent component.

Application in the Veterinary Medicine. The treatment of kolpitis in female dogs by the present combinations proved similarly effective as compared to human treatment.

Treatment of the inflamed glandula Bartholini by drainage and injected solution of a composition of the invention instead of simple incision or marsupialization. Other similar applications are also possible inside the body.

Combined (local and systemic) treatment: (a) In pregnancy to prevent intrauterine Chlamydia trachomatis infection causing pneumonia or perhaps blindness (see Milánkovits, Márton: The Possible Role Of Non Trachoma Chlamydia Trachomatis Serovars In Intrauterine Blindness, 12th Congress of the European Association of Gynecologists & Obstetricians, 25th-28th Jun. 1997, Trinity College, Dublin, Ireland.); and (b) In cases of infections of sportswomen (mainly carriers) to improve their physical abilities and fitness.

The compositions according to the invention were tested on 300-400 cases, with 300-400 controls. In all cases, the full microbiological examination included Chlamydia trachomatis and the Mycoplasmas.

Preparations of the compositions according to the invention can be carried out by methods known in the art for the preparation of such compositions.

The following experimental results demonstrate the unexpected efficacy of the compositions of the present invention, and set forth exemplary compositions indicative of the many possible species combinations useful in the treatment of various maladies. The examples will serve to further typify the nature of the invention, but should not be construed as a limitation on the scope thereof, which is defined solely by the appended claims.

### Example 1

309 Patients
Seven days treatment

| | |
|---|---|
| Chloramphenicol: | 0.10 g |
| Sulfadimidin: | 0.10 g |
| Nystatin: | 0.10 g |
| Metronidazole: | 0.40 g |
| Massa Polyoxaethenum: | 2.00 g |

Completely treated: 306
Remained the same: 3
Worsened: 0

### Example 2

90 Patients
Seven days treatment

| | |
|---|---|
| Ciprofloxacin: | 0.04 g |
| Sulfadimidin: | 0.10 g |
| Nystatin: | 0.10 g |
| Metronidazole: | 0.40 g |
| Massa Polyoxaethenum: | 2.00 g |

Completely treated: 89
Remained the same: 1
Worsened: 0

### Example 3

77 Patients
Seven days treatment

| | |
|---|---|
| Ampicillin: | 0.20 g |
| Sulfadimidin: | 0.10 g |
| Nystatin: | 0.10 g |
| Metronidazole: | 0.40 g |
| Massa Polyoxaethenum: | 2.00 g |

Completely treated: 75
Remained the same: 2
Worsened: 0

### Example 4

60 Patients
Seven days treatment

| | |
|---|---|
| Sulfadimidin: | 0.10 g |
| Nystatin: | 0.10 g |
| Metronidazole: | 0.40 g |
| Massa Polyoxaethenum: | 2.00 g |

Completely treated: 52
Remained the same: 8
Worsened: 0

### Example 5

19 Patients
Seven days treatment

| | |
|---|---|
| Neomycin: | 0.10 g |
| Sulfadimidin: | 0.10 g |
| Nystatin: | 0.10 g |
| Metronidazole: | 0.40 g |
| Massa Polyoxaethenum: | 2.00 g |

Completely treated: 18
Remained the same: 1
Worsened: 0

### Example 6

10 Patients
Seven days treatment

| | |
|---|---|
| Polymixin: | 0.05 g |
| Sulfadimidin: | 0.10 g |
| Nystatin: | 0.10 g |
| Metronidazole: | 0.40 g |
| Massa Polyoxaethenum: | 2.00 g |

Completely treated: 9
Remained the same: 1
Worsened: 0

### Example 7

12 Patients
Seven days treatment

| | |
|---|---|
| Semicillin: | 0.20 g |
| Sulfadimidin: | 0.10 g |
| Nystatin: | 0.10 g |
| Tinidazole: | 0.40 g |
| Massa Polyoxaethenum: | 2.00 g |

Completely treated: 11
Remained the same: 1
Worsened: 0

### Example 8

10 Patients
Seven days treatment

| | |
|---|---|
| Semicillin: | 0.20 g |
| Sulfadimidin: | 0.10 g |
| Clotrimazole: | 0.10 g |
| Metronidazole: | 0.40 g |
| Massa Polyoxaethenum: | 2.00 g |

Completely treated: 9
Remained the same: 1
Worsened: 0

### Example 9

10 Patients
Seven days treatment

| | |
|---|---|
| Semicillin: | 0.20 g |
| Sulfadimidin: | 0.10 g |
| Natamycin: | 0.30 g |
| Metronidazole: | 0.40 g |
| Massa Polyoxaethenum: | 2.00 g |

Completely treated: 9
Remained the same: 1
Worsened: 0

### Example 10

10 Patients
Seven days treatment

| | |
|---|---|
| Chloramphenicol: | 0.10 g |
| Nystatin: | 0.10 g |
| Metronidazole: | 0.40 g |
| Massa Polyoxaethenum: | 2.00 g |

Completely treated: 8
Remained the same: 2
Worsened: 0

### Example 11

10 Patients
Seven days treatment

| | |
|---|---|
| Semicillin: | 0.20 g |
| Natamycin: | 0.30 g |
| Metronidazole: | 0.40 g |
| Massa Polyoxaethenum: | 2.00 g |

Completely treated: 7
Remained the same: 3
Worsened: 0

### Example 12

8 Patients
Seven days treatment

| | |
|---|---|
| Chloramphenicol: | 0.10 g |
| Sulfadimidin: | 0.10 g |
| Nystatin: | 0.10 g |
| Metronidazole: | 0.10 g |
| Massa Polyoxaethenum: | 2.00 g |

Completely treated: 7
Remained the same: 1
Worsened: 0

### Example 13

Systemic administration (per os)
9 Patients
Seven days treatment

| | |
|---|---|
| Ciprofloxacin: | 750 mg/day (500 mg + 250 mg) |
| Ketoconazole: | 400 mg/day (200 mg + 200 mg) |
| Tinidazole: | 1000 mg/day (500 mg + 500 mg) |

Completely treated: 8
Remained the same: 1
Worsened: 0

The above examples demonstrate the interchangeability of specific species within the respective genera antibacterial agent, nitroimidazole, and antifungal agent effective against a Candida species, while maintaining the efficacy of the various compositions of the present invention. Examples 10 and 11 illustrate the high level of effectiveness of even three component compositions of the present invention. Such effective experimental results are particularly unexpected when contrasted with the treatment efficacy using a single active ingredient. Such monotherapy treatments result in less than 60% complete recovery (e.g. for bacterial vaginosis).

The compositions demonstrate significantly reduced treatment periods compared to application of these components separately. Previous treatment of the patients comprising the above test groups had failed with standard local administration of:

| Agent | Duration of Treatment |
|---|---|
| Klion (metronidazole) | 10 days |
| Klion D | 10 days |
| Pimafucin (natamycin) | 20 days |
| Canesten (clotrimazol) | 8-10 days |

For example, it has been found that no benefit is realized through repeated 2000 mg doses of metronidazole administered alone (see Carey J C, Klebanoff M A, et al. Metronidazole to Prevent Pre-term Delivery in Pregnant Women with Asymptomatic Bacterial Vaginosis, New England Journal of Medicine 2000; 342:534-40). In a further aspect of the present invention, it has been found that the respective effectiveness of a nitroimidazole and a sulfonamide when utilized alone is enhanced when combined. As such, an unexpected success rate of over 80% against bacterial vaginosis is achieved through the combination of a sulfonamide and a nitroimidazole. In the compositions of the invention the sulfonamides or nitroimidazoles are unexpectedly strongly effective against most of the prokaryotic cells and the unhealthy mammalian (eukaryotic) cells too by causing their early death. (Unhealthy means infected cells and tumor cells). The TP53 gene encodes P53. One of its guardian functions is to stop cells from replicating damaged DNA. Normal cells with damaged DNA arrest at a checkpoint at the G1/S stage of the cell cycle until the damage is repaired, but unhealthy cells do not. Probably related to not normal “unhealthy cells” is a crucial role of p53 in programmed cell death (apoptosis). In the compositions of the present invention the (a) nitroimidazoles (mutagenic effects) and (b) sulfonamides (antifolates-teratogen effects) caused damage in DNA respectively, and therefore unexpectedly increase the effectiveness of the other ingredient or ingredients.

(a) Early work established that metronidazole inhibits DNA synthesis in T. vaginalis and Clostridium bifermentans and causes degradation of existing DNA in the latter microorganism. These findings are consistent with the antimicrobial and mutagenic effects of metronidazole. Once the drug has diffused into the cells, the nitro group accepts electrons from electron-transport proteins with sufficiently low negative redox potentials, e.g. flavoprotein in mammalian cells and ferredoxins or their equivalent in protozoa and bacteria (see GOODMAN-GILMAN page 996).

(b) Sulfonamides are structural analogs and competitive antagonists of para-aminobenzoic acid (PABA), and thus prevent normal utilization of PABA for the synthesis of folic acid. One of the most active antibacterial agents that exert a synergistic effect when used with a sulfonamide is trimethoprim. The simultaneous administration of these agents thus introduces sequential blocks in the pathway of the synthesis of tetrahydrofolate from precursor molecules (see GOODMAN-GILMAN page 1058). Sulfonamides do not affect healthy mammalian cells. Antifolates occupy a special place in antineoplastic chemotherapy, e.g. Methotrexate—its toxicity includes teratogenesis.

Examples in the present application supporting (a) and (b) are as follows:

(1) Precancerous cells died: Following the treatment with the present compositions, the cytological findings of Papanicolau smears improved from P3 to P2 or P1. The two cases of P4 improved to P2 and P1 respectively. The precancerous or cancerous cells disappeared. They died because the mutagenic effect can damage the DNA of the cells but can not repair DNA and thus cure the cells; and

(2) Infected cells of Condyloma acuminatum died (the infection caused by the human papilloma virus): Probably because of the early death of the host cells, the formation of mature infectious progeny virus is not possible, perhaps the steps of the viral replicative cycle could not be successfully completed.

It has been observed through experimental data that the antibacterial or antitumor effect of sulfonamide or nitroimidazole standing alone is greatly enhanced when coupled with the other antibacterial ingredient or ingredients of the present compositions. Therefore, such an unexpected increased effectiveness of sulfonamide or nitroimidazole when incorporated into the compositions of the present invention forms an additional embodiment and aspect of the present invention.

Sulfonamides are structural analogs and so competitive antagonists of para-aminobenzoic acid (PABA): C₇H₇NO₂. They prevent the synthesis of folic acid and this prevention results in folic acid antagonist effect. The term sulfonamide is employed herein as a generic name for derivates of para-aminobenzenesulfonamide (sulfanilamide): C₆H₈N₂O₂S (e.g. sulfamethoxazole: C₁₀H₁₁N₃O₃S. The minimal structural prerequisites for antibacterial action are all embodied in sulfanilamide itself. The SO₂NH group is not essential as such, but the important feature is that the sulfur is directly linked to the benzene ring. The para-NH₂ group (the N of which has been designated as N4) is essential and can be replaced only by such radicals as can be converted in vivo to a free amino group. Substitutions made in the amide NH₂ group (the N of which has been designated as N1) have variable effects on antibacterial activity of the molecule. Substitution of heterocyclic aromatic nuclei at N yields highly potent compounds, (e.g. sulfamethoxazole).

An additional aspect and embodiment of the present invention is nitroimidazole substitution made in the amide NH₂ group of sulfanilamide or the same substitution(s) made in the single ring of the lipid soluble antifolate trimetrexate or made on methotrexat. The effectiveness of the antifolates such as the p-Aminobenzamide mimics and the Pteridine mimics on prokaryotic and eukaryotic cells is dosage dependent and also depends on the general health-condition of the cells

The unexpectedly increased effectiveness provided by the combined application of sulfadimidine and metronidazole, as it was demonstrated in the granted U.S. Pat. No. 6,432,935, partly can be explained by the antifolic acid effect of the sulfonamides and the simultaneous cytotoxic effect of the nitroimidazoles strengthening each other when are acting together on bacteria or on the weakened ill human cells. The possible further explanation of the increased effectiveness could be that both of them have antitumor effectiveness as it is summarized briefly below: In the 1940s scientists discovered that sulfanilamide blocked the activity of an enzyme called carbonic anhydrase. In 1993, Teicher et al. reported that carbonic anhydrase (CA) inhibitors, as part of a chemotherapy regimen, enhanced the chemotherapeutic drug effects and suppressed the tumor cells. (In 2008. Supuran Claudiu T reported that the carbonic anhydrase inhibitors beside their antitumor effect have antimicrobial effects too because they are targeting CAs from pathogenic organisms such as Helicobacter pylori, Mycobacterium tuberculosis, Plasmodium falciparum, Candida albicans, etc.) The nitroimidazoles also have certain anticancer effects. In 1989 Trujillo J M et al evaluated metronidazole as a single agent or in combination with cis-diamminedichloroplatinum and 1-beta-arabinofuranosylcytosine for its cytotoxic effects on five established human colon carcinoma cell lines. Metronidazole enhanced the synergism resulting from the combination of the two antitumor agents and alone produced dose-dependent cytotoxic effect.

When the 1-(2 hydroxy-ethyl)-2-methyl-4-/(4′amino-phenyl-sulfonyl)amino/-5-nitroimidazole, one of the sulfonamides (or the present sulfonamide or its derivatives or a sulfanilamide molecule respectively substituted similarly to Prontosil but by a nitroimidazole molecule) described below has entered into a cell, then its metabolism occurring at the place of the substitution results in the originally (before substitution) separate two molecules of acting agents (sulfanilamide and nitroimidazole). Application of the present molecules in the combinations of U.S. Pat. No. 6,432,935 provides a very important advantage, namely the simultaneous entering into the cells of two active agents of the combinations. Naturally, this advantage is present when any of the present molecules is applied alone as a monotherapy.

Direct and Indirect Anti HIV/AIDS Effectiveness. The international recognition of U.S. Pat. No. 6,432,935 was expressed through prizes awarded for the anti HIV/AIDS effectiveness of such pharmaceutical compositions, in Moscow(1), Geneva(2), and Warsaw(3) 1.) VIII. Moscow International Salon of Innovations and Investments (inventions, investment-attractive innovations, high technologies) 3-6 Mar. 2008, All-Russian Exhibition Center, Pavilion 69, Moscow. 2.) 36. International Exhibition of Inventions, New Techniques and Products in Geneva, on 2-6 Apr. 2008. 3.) IWIS—International Warsaw Inventions Show 2008 on Jun. 5, 2008. Palace of Culture & Science.

The importance of the combined treatment for bacterial vaginosis was demonstrated in a comparative, in vivo, human, prospective, single blind, clinical and microbiological diagnoses based and randomized study (see Milankovits, M., Baksay, L., Plachy, J.: Comparative study of combined local treatment (sulfadimidine, metronidazole and nystatin) and the standard monotherapy in uncomplicated bacterial vaginosis. Orv Hetil. 2002 Dec. 22; 143(51):2835-40.)) and later presented with accomplished observations (see Milankovits, M.: The effect of combined antimicrobial treatment on prokaryotic cells and on certain groups of eukaryotic cells. —Oral presentation. World and Ehrlich Conference, Nürmberg, Sep. 9-11, 2004.) The effectiveness of the presently available and applied monotherapies for bacterial vaginosis is less than optimal (see Erica Weir: Bacterial vaginosis: more questions than answers CMAJ 2004; 171: 448). Only combined treatment can provide optimal efficacy (see Milankovits, M.: More effective treatment for bacterial vaginosis. CMAJ, eLetters for Weir. 4 Mar. 2005). The more effective combined treatment often times prevents the development of chronic diseases (see Milankovits, M.: Infections and chronic diseases. Nov. 11, 2005. Comments on: The neglected epidemic of chronic disease The Lancet—Vol. 366, Number 9496, 29 Oct. 2005, Pages 1514).

An additional aspect and embodiment of the present invention is the application of the new molecules in Photomedicine. The compositions of U.S. Pat. No. 6,432,935 were applied successfully by Sommer. (see Andrei P. Sommer Antiinfectives and Low-Level Light: A New Chapter in Photomedicine and Laser Surgery. Jun. 1, 2007, 25(3): 150-158. doi:10.1089/pho.2007.2058.

The combined treatment is especially important for the treatment of infertility (of infectious origin) and dental diseases.

The following examples demonstrate the effectiveness of a pharmaceutical composition containing a sulfonamide and/or a nitroimidazole with and without trimethoprim.

### Example 14

6 Patients
Seven days treatment

| | |
|---|---|
| Sulfamethoxazol: | 0.10 g |
| Trimethoprim: | 0.02 g |
| Metronidazole: | 0.20 g |
| Massa Polyoxaethenum: | 2.00 g |

Completely treated: 5
Remained the same: 1
Worsened: 0

### Example 15

7 Patients
Seven days treatment

| | |
|---|---|
| Sulfadimidin: | 0.10 g |
| Metronidazole: | 0.20 g |
| Nystatin: | 0.10 g |
| Natamycin: | 0.20 g |
| Massa Polyoxaethenum: | 2.00 g |

Completely treated: 6
Remained the same: 1
Worsened: 0

Before treatment using the present combinations, vaginal bacterial samples were taken with cotton swabs (for culture and gram stain). The most common microorganisms found were E. Coli, Enterococcus faecalis, B-group streptococci, Candida albicans, (Urea plasma) Ureaplasma urealyticum, Mycoplasma hominis, Gardnerella, Trichomonas vaginalis, Chlamydia trachomatis, and, to a lesser extent, Staphylococci, Proteus, Klebsiella, Haemophylus, etc. Antibiotic sensitivity was examined as well. After taking samples, patients were treated with the above-listed combinations of the present invention.

In addition to providing an unexpected increase in efficacy of each individual component when utilized in the combinations of the present invention, a significantly lower amount of each active ingredient is needed in the present invention as compared to typical monotherapy formulations.

The effective treatment of bacterial vaginosis is especially important because of its serious clinical implications and morbidity such as post-hysterectomy vaginal cuff cellulitis, plasmacell endometritis. In pregnant women, such clinical implications include amniotic fluid infection, clinical chorioamnionitis, postpartum endometritis, premature rupture of the membranes, pre-term delivery, and low birth weight.

The compositions of the present invention further displayed unexpected anti-viral activity. For example, in cases where the treatment of Condyloma acuminatum (caused by the human papilloma virus) failed, treatment using the compositions of the present invention along with local Podophyllin treatment proved to be permanently successful. Moreover, it has been observed that patients with frequently recurring herpes genitalis became permanently free of symptoms following treatment with the preparations of the present invention in 75% of the cases tested.

The following additional examples are contemplated by the present invention for use in treatment compositions:

Combination I:

| | | |
|---|---|---|
| Chloramphenicol | 0.08 | g |
| Sulfadimidin | 0.20 | g |
| Clotrimazol | 0.15 | g |
| Metronidazole | 0.40 | g |
| Massa polyoxaethenum | 2.0 | g |

Combination II:

| | | |
|---|---|---|
| Chloramphenicol | 0.1 | g |
| Sulfadimidin | 0.1 | g |
| Clotrimazol | 0.1 | g |
| Metronidazole | 0.40 | g |
| Massa polyoxaethenum | 2.0 | g |

Combination III:

| | | |
|---|---|---|
| Unasyn | 0.1 | g |
| Sulphadimidin | 0.1 | g |
| Nystatin | 0.1 | g |
| Metronidazole | 0.4 | g |
| Massa polyoxaethenum | 2.0 | g |

Combination IV:

| | | |
|---|---|---|
| Augmentin | 0.1 | g |
| Sulphadimidin | 0.1 | g |
| Nystatin | 0.1 | g |
| Natrium-tetraborat | 0.05 | g |
| Metronidazole | 0.4 | g |
| Massa polyoxaethenum | 2.0 | g |

Combination V:

| | | |
|---|---|---|
| Oxacillin | 0.1 | g |
| Sulphadimidin | 0.1 | g |
| Nystatin | 0.1 | g |
| Metronidazole | 0.4 | g |
| Massa polyoxaethenum | 2.0 | g |

Combination VI:

| | | |
|---|---|---|
| Cefaclor (Ceclor) | 0.05 | g |
| Sulphadimidin | 0.1 | g |
| Nystatin | 0.1 | g |
| Metronidazole | 0.4 | g |
| Massa polyoxaethenum | 2.0 | g |

Combination VII:

| | | |
|---|---|---|
| Gentamicin | 0.05 | g |
| Sulphadimidin | 0.1 | g |
| Nystatin | 0.1 | g |
| Metronidazole | 0.4 | g |
| Massa polyoxaethenum | 2.0 | g |

Combination VIII:

| | | |
|---|---|---|
| Clarithromycin (Klacid) | 0.1 | g |
| Sulphadimidin | 0.1 | g |
| Nystatin | 0.1 | g |
| Metronidazole | 0.4 | g |
| Massa polyoxaethenum | 2.0 | g |

Combination IX:

| | | |
|---|---|---|
| Chloramphenicol | 0.1 | g |
| Sulphadimidin | 0.1 | g |
| Nystatin | 0.1 | g |
| Natrium-tetraborat | 0.05-0.10 | g |
| Metronidazole | 0.4 | g |
| Massa polyoxaethenum | 2.0 | g |

Combination X:

| | | |
|---|---|---|
| Clindamycin | 0.05 | g |
| Natrium-tetraborat | 0.05 | g |
| Sulphadimidin | 0.1 | g |
| Nystatin | 0.1 | g |
| Metronidazole | 0.4 | g |
| Massa polyoxaethenum | 2.0 | g |

The present combination can be used in applications such as suppositories, ointments, talc powder, solution, painting solutions, vaginal drops, or impregnated tampons.

For systemic treatment situations, the preparations contain the combination of the active ingredients in the same ratio as described above for local treatment.

Ointment preparations contain the combination of the active ingredients in the same ratio as in the vaginal suppository, together with ointment base, with yellow Vaseline and other components known per se, if required. This preparation is especially effective in case of tissue damage (diabetes mellitus, burning, etc.). The talc powder preparation contains the active ingredient combination in solid form, with carriers such as talc, etc. The painting solutions and vaginal drops are prepared with an appropriate organic solvent. The vaginal drop solutions are useful in pediatric gynecology; but can also be used for adults in adequate doses.

Another aspect of the invention is the combined treatment or monotherapy applying an extraordinary amount of Rocephine (Ceftriaxone) for the treatment of Amyotrophic lateral sclerosis (ALS). ALS, also known as Lou Gehrig's disease, sometimes called Maladie de Charcot, is a progressive wasting away of certain nerve cells of the brain and spinal column called motor neurons, a devastating condition characterized by progressive muscle wasting, inanition, respiratory failure, and death within approximately 2 to 5 years of onset. ALS is among the most common neuromuscular conditions, with an overall prevalence in the world of approximately 5 to 7 cases/100,000 population and people of all races and ethnic backgrounds are affected. One to two people per 100,000 develop ALS each year. Between 5% and 10% of ALS is genetic. (See Distad B J, “Drug therapy in amyotrophic lateral sclerosis.” Phys Med Rehabil Clin N Am. 2008 August; 19(3):633-51, xi-xii.) The state of the art and the tendency of future research work regarding the treatment and pathomechanism of ALS are reflected in the recent publication of Nicholas J. Maragakis (Maragakis N J.: Rethinking a drug treatment failure on a traditional ALS target. Exp Neurol. 2009 April; 216(2):254-7) The theoretical basis of the present invention is the following: the well known antimicrobial agents are usually effective in higher dosage against ill human cells and probably in even much higher dosages against healthy cells too. The combined application of active agents facilitates and improves the effectiveness of the applied single agents, therefore the usual or smaller dosage of the single agents provides the proper efficacy in the combinations of the invention but there is a strong possibility that when a proper agent is administered alone in properly high dosage, the desired effect may also be reached alone by monotherapy.

The observations mentioned above are based on the experience on Rocephine (a broad-spectrum cephalosporin antibiotic with a very long half-life and high penetrability to meninges, eyes and inner ears, its generic name being Ceftriaxone) treatment for a case having the familial, inherited form of ALS. One inherited, early and successfully treated case is not enough to prove the effectiveness of the suggested new treatment but the success is an encouraging and inspiriting fact to examine it as a possible treatment for such a presently incurable disease as ALS. (Late treatment of ALS does not appear to be effective, likely because the disease has progressed to the point of damaging or destroying too many cells for recovery.) In another example, speaking difficulties derived from ALS had disappeared for three weeks following a 12-day regimen of Doxycyclin (per os 2×100 mg/day). Presently, Riluzole is the primary drug used to treat amyotrophic lateral sclerosis. “This is a modest effect, but before Riluzole there was no therapy for ALS at all. Riluzole prolonged survival on average by about three months”. (FDA APPROVES FIRST DRUG FOR LOU GEHRIG'S DISEASE, Food and Drug Administration-P95-11, Dec. 12, 1995) Rocephine is an effective antibiotic and enough even in just one single intramuscular dosage to cure gonorrhoeal infection. It can not be administered intramuscularly in high dosage due to the potential for muscular damage. The properly high dosage for ALS can be administered only intravenously. The treatment should start at the first appearance of the characteristic features and complaints of the disease. The dosage is 100 mg/kg bodyweight/day (50 mg/kg in a morning infusion (intravenous drip) and an evening infusion respectively.) In case of 80 kg or 100 kg bodyweight the daily amount of Rocephin is 8.0 or 10.0 grams respectively. The treatment starts with two, fortnight long treatments, with a few days pause between them. Around the end of the first week of high starting dosage ceftriaxone treatment, side effects may appear as a result of cumulation of ceftriaxone. In such cases, the further dosages must be decreased. The second or the third and the following infusions contain just half of the initial dosage. The duration of treatment is around six months and the whole amount of Rocephine could be up to about 500 grams. The result was that the progression of the disease stopped one and a half decades ago, but diabetes mellitus, more exactly metabolic syndrome, as well as autoimmune alterations such as Hashimoto's thyreoiditis and myocardial infarction have developed. The first month of combined treatment following inferior myocardial infarction and percutaneous coronary intervention by a statin, clopidogrel and Endeavor™ stents was followed by loss of muscles, swallowing and speaking difficulties, itching, and so on, but the relatively prompt and intensive ceftriaxone treatment diminished the swallowing complaints and muscle fibrillations and stopped the other complaints again. The lost muscles did not recover. The two systematically administered medicines, and the locally applied third medicine (Endeavor DES stents, 3 pieces, 30+30+12=72 mm long altogether) have an important common feature, namely each of them has cytotoxic effect. The medicines have different mechanisms of action, and they may therefore potentiate the effectiveness of each other. The cytotoxic effect could play an important role in eliciting ALS. The heart complaints and dyspnoea were much more severe after the percutaneous coronary intervention and the connected combined treatment mentioned above than before it during the first 39 days after myocardial infarction. The percutaneous coronary intervention was performed 39 days after the myocardial infarction and after the normalization of cardiac troponin-I serum level. The severe heart complaints gradually diminished during the ceftriaxone treatment. It shows the likely involvement of the special muscles of the heart in the pathological process of ALS. Ceftriaxone has neuroprotective effectiveness and might also have direct or indirect cardiomyoprotective effectiveness in ALS. The neuroprotective effectiveness may likely involve not just motoneurons, but also vegetative neurons (autonomic nervous system) according to the observations. During the ceftriaxone drips, less medicines were enough than directly before them to treat the hypertension and keep the normal tension. (Forty four years earlier hyperkinetic circulation syndrome was diagnosed.) ALS may be a multisystem degenerative disorder with a very long latent period of time, and the autonomic disregulation may be a part of the complex degenerative process.) The application of an ointment composition according to U.S. Pat. No. 6,432,935 provided the possibility of continuous application of the same intravenous cannula for a longer period of time (38 days) and for 58 drips (136 g) of ceftriaxone during this time. Five weeks later, because of mild relapse, a further 168 g of ceftriaxone was administered, 2 grams, twice a day. Even in the case of this very early treatment the necessary amount of ceftriaxon was above 300 g (304 g) and this amount does not necessarily constitute the end of the treatment. The fact that ceftriaxone administered in high dosages and in extraordinary high whole amounts was able to stop the progression of familial ALS again, a second time after nearly 18 years, shows that presently the ceftriaxone treatment is the most effective treatment for ALS.

ALS occurred on the island of Guam with unusually high incidence rates for many years but began to disappear after WW II and there is a concurrent surge in the prevalence of diabetes. (See Robert L Haddock1 and Kwang-Ming Chen2 “AMYOTROPHIC LATERAL SCLEROSIS AND DIABETES ON GUAM: CHANGING PATTERNS OF CHRONIC DISEASE IN AN ISLAND COMMUNITY” Southeast Asian J Trop Med Public Health. September 2003; 34(3):659-661. The high spouse correlation will be a challenge to environmental epidemiology of ALS. (See Hemminki K et al. “Familial risks for amyotrophic lateral sclerosis and autoimmune diseases.” Neurogenetics. 2008 Dec. 17, and Corcia P. et al.: A clustering of conjugal amyotrophic lateral sclerosis in southeastern France.: Arch. Neurol. 2003 April; 60[4]:553-7.) Corcia P. et al. describe nine couples in which both spouses were affected by ALS. In spite of the important observations of Corcia P. et al., it is too early at this stage to put the question of whether ALS might be sexually transmitted, but in the near future, such a question may need to be addressed through possible horizontal and vertical spread. The introduction of new categories such as incomplete and complete ALS, as a new approach, might be helpful in the early diagnosis of ALS. It might be that many times the spouse of the ALS subject had “incomplete” ALS and therefore she or he was not diagnosed. ALS can be incomplete quantitatively and/or qualitatively. The quantitative aspect is on the basis of the severity of the alterations, and the qualitative aspects are on the basis of the number of the characteristic alterations. In the early stage, the disease likely always appears in its incomplete form. The “El Escorial Criteria for Motor Neuron Disease-Modified version-Nov. 5, 2004” and “El Escorial Criteria for ALS-Jul. 13, 2008” must be fulfilled. ALS is a progressive disease. During the time while the clinically possible ALS develops into clinically definite ALS its treatability by ceftriaxone can be lost. The observation alone does not seem to be enough. The early treatment should start from the “possible” stage in case of growing suspicion. The results of the genetic research work are promising, however it is difficult to answer the question if the mutations /SOD1, discovered in 1993; a new gene, ALS6 (Fused in Sarcoma) discovered most recently, etc./ found in the minor part of patients having familial ALS are the causes or just the consequences of ALS. In the larger part of familial ALS, patients and in the rest of the ALS patients (ninety percent, so called sporadic cases), the mutations are not present. The findings of Tan C F et al. indicate that the histological and molecular pathology of SALS can occur as a phenotype of FALS without SOD1 mutation. (Tan C F, Eguchi H, Tagawa A, et al.: TDP-43 immunoreactivity in neuronal inclusions in familial amyotrophic lateral sclerosis with or without SOD1 gene mutation. Acta Neuropathol. 2007. May; 113(5):535-42.) There are no data available regarding the prevalence of the mutations mentioned above in the healthy population.

The mutations might be the results of the complicated host-pathogen interactions during the passage of a causing agent in the sensitive cases. The possibility of vertical and horizontal spread supports the hypothesis of infection. Not only the genetic diseases but the infectious diseases can also involve members of a family and aggregate within kindreds. In both familial and sporadic ALS, the disease symptoms and pathology are the same. Further research is necessary to clarify the cause of ALS. “The onset of ALS may be so subtle that the symptoms are frequently overlooked.” (National Institute of Neurological Disorders and Stroke (NINDS) Amyotrophic Lateral Sclerosis Fact Sheet.) The diversity of alterations also contributes to the difficulties of early diagnosis. Even in the case of the same person the second onset, after nearly 18 years, was remarkably different from the first onset. The mild difficulty with speech, swallowing, and muscle atrophy of the hands were present in both onsets, however further visible loss of dorsal interosseus muscles and palmar interosseous muscle involvement was observed at the second onset. The weight loss experienced in the first onset was nine kilogram during ten days. The loss of facial muscles of the right part of the face resulted in asymmetry of the two halves of the face. Frequent fasciculations occurred involving both the lower and upper limbs but rarely the muscles around the mouth too. Dominant muscle atrophy in the thenar as compared to the hypothenar complex has occurred, mainly on the right hand, likely because of corticomotoneuronal involvement. (Weber Markus, Eisen Andrew, Stewart Heather, Hirota Nobuyuki: The split hand in ALS has a cortical basis. Journal of the neurological sciences ISSN 0022-510X. Congrés International Symposium on Amyotrophic Lateral Sclerosis/Motor neuron Disease No 10, Vancouver, CANADA (Nov. 15, 1999) 2000, vol. 180, no 1-2 (115 p.) (34 ref.) pp. 66-70.) Continuous cramps in the right calf were painful, involuntary, lasted several months and resulted in loss of muscles. The decrease in its circumference was 1.5 centimeters compared with the left side. Second time the fasciculations were rare, but severe heart complaints and dyspnoea appeared. The loss of weight was 4 kilogram during 6 weeks, but after the ceftriaxone treatment just half a kilogram. The findings of Borrelia burgdorferi serology were negative in 1991 and 2009 respectively. The further details are medically important. A group of neurophysiologists suggested the incorporation of electrophysiological signs of LMN dysfunction equally to clinical signs and the interpretation of the presence of fasciculation potencials in the diagnosis of ALS at a conference on Awaji Island in 2006. Their aim was to define the possibility of earlier diagnosis with a higher certainty level. The extensive laboratory, imaging and electrophysiological studies are usually non-diagnostic but can support the clinical diagnosis especially in doubtful cases. The indication of the electrophysiological examination however naturally depends on a previous and hopefully early decision based on the clinical basis. A very detailed score system, suitable for quick, computerized, thorough evaluation of the alterations and follow the progression, should be worked out to recognize the onset of the disease as early as possible. The duration of the early stage depends on different factors. The transmission of ALS by transfusion or other hematogen route seems to be possible. The special application of ceftriaxone may not cure ALS patients, but it retards the progression of the disease or cease its progression for a longer period of time (even for decades), and so can provide a seemingly healthy life. The properly early treatment can be provided if the members (each of them, with or without genetic relation) of the involved families (having at least one ALS victim member) have been informed about the early features of ALS and in case of suspicion (based on the identification of early alteration(s)) the confirming examination and ceftriaxon treatment can start without any delay. (Suspicion emerges when e.g. a teenager member of the involved family has muscle fasciculations, positive Babinsky reflex and sudomotor dysfunction. /personal observation/) The evaluation of these results could be the subject of a study. Furthermore, it seems to be important to inform and examine the members of the involved families before they have any complaints and analyze the results in a prospective study. It is important to take into consideration that there is a strong possibility that statins (and possibly some other medicines) can elicit ALS, probably mainly in the members of such families. In the case mentioned above, it is likely that rosuvastatin did elicit ALS after a 25 day, 20 mg/day treatment regimen. The possible role of this and other medicines could be cleared by examinations in mouse models (studies in the standard murine model of ALS). The WHO Foundation Collaborating Centre for International Drug Monitoring (Uppsala Monitoring Centre [UMC]) has received many individual case safety reports (ICSRs) associating HMG-CoA reductase inhibitor drug (statin) use with the occurrence of amyotrophic lateral sclerosis (ALS)-like syndrome. Of the total of 172 ICSRs on this reported term, 43 were related to statins, of which 40 were considered further: all but one case was reported as ALS. In 34/40 reports a statin was the sole reported suspected drug. (Edwards, I Ralph; Star, Kristina; Kiuru, Anne: Statins, Neuromuscular Degenerative Disease and an Amyotrophic Lateral Sclerosis-Like Syndrome: An Analysis of Individual Case Safety Reports from Vigibase; Drug Safety. 30(6):515-525, 2007.) A prospective study could evaluate the effectiveness of ceftriaxone treatment for “ALS-like” syndrome. Another group of target patients possibly belongs to “incomplete” ALS and the patients having muscle fibrillations and the loss of considerable muscle and weight. Such patients may die because of e.g. myocardial infarction before the complete ALS develops. The disease can start with severe anxiety, heart complaints, etc., and also before the mass loss of muscles, muscle fibrillations and spasms. The sooner that treatment begins, the smaller the necessary whole amount of ceftriaxone. The patients are vulnerable and the invasive diagnostic and therapeutic methods possibly should be avoided. Late treatment of ALS could potentially worsen the condition of the patient. Several studies have examined effectiveness of ceftriaxone treatment for ALS over the past 17 years. (General info: Treatment: Ceftriaxone; Treatment class: Small molecule; Conclusions: No effect.) The following are among them: 1.) Couratier P, Vallat J M, Merle L, Preux P M, Hugon J.: Report of six sporadic cases of ALS patients receiving ceftriaxone. Therapie. 1994 March-April; 49(2):146. 2.) Carelli V, Liguori R, Cordivari C, Bianchedi G, Montagna P.: Ceftriaxone is ineffective in ALS. Ital J Neurol Sci. 1994 February; 15(1):66. 3.) Norris F H.: Ceftriaxone in amyotrophic lateral sclerosis. Arch Neurol. 1994 May; 51(5):447. 4.) Carod Artal F J, Pérez López-Fraile I, Gracia Naya M, Girón Mombiela J A.: Failure of empirical treatment with ceftriaxone in motor neuron disease. Neurologia. 1994 January; 9(1):29-31. 5.) Comment on: Neurologia. 1994 January; 9(1):29-31. Gil Llano J R, Casado Naranjo I.: Lack of improvement of motoneuron disease with ceftriaxone.: Neurologia. 1994 May; 9(5):205-6. 6.) Couratier P, Vallat J M, Merle L, Preux P M, Hugon J.: Report of six sporadic cases of ALS patients receiving ceftriaxone. Therapie. 1994 March-April; 49(2):146. 7.) Smith L G.: Improvement of patient with amyotrophic lateral sclerosis given ceftriaxone. Lancet. 1992 Jun. 6; 339(8806):1417. 8.) Comment on: Lancet. 1992 Jun. 6; 339(8806):1417. Smith L G.: Failure of ceftriaxone for amyotrophic lateral sclerosis. Lancet. 1992 Aug. 8; 340(8815):379. 9.) Comment on: Lancet. 1992 Jun. 6; 339(8806):1417. Robberecht W.: Lack of improvement with ceftriaxone in motoneuron disease. Lancet. 1992 Oct. 31; 340(8827):1096-7. (Conclusions: improvement from drug, later declined in subsequent publication). The reasons of failure are very likely the small amounts of the administered ceftriaxone and the possibly not early start of treatment.

Presently, the answer for the question “Should I ask my doctor to prescribe ceftriaxone (Rocephin) or another similar antibiotic for me to take?” may be the following: “No, the researchers doing this study and other ALS clinical experts agree that it is premature for patients to take ceftriaxone for ALS.” (See: (Roberta Friedman, Ph.D., ALSA Research Department Information Coordinator.) Over the past 15 years, more than 25 potential drugs have been tested in randomized controlled trials. Despite this progress, we have no medications other than riluzole. (Mitsumoto H. A strategy to develop effective ALS therapy; Brain Nerve. 2007 April; 59(4):383-91). A study for the use of ceftriaxone in ALS for compassionate care has been completed in the United States “Compassionate Use of Ceftriaxone in Patients With Amyotrophic Lateral Sclerosis (ALS)” (ClinicalTrials.gov Identifier: NCT00718393). Significant progress in understanding the cellular mechanisms of motor neuron degeneration in amyotrophic lateral sclerosis (ALS) has not been matched with the development of therapeutic strategies to prevent disease progression. (Vincent A M, Sakowski S A, Schuyler A, Feldman E L. Strategic approaches to developing drug treatments for ALS. Drug Discov Today. 2008 January; 13(1-2):67-72.) There is a currently ongoing study in the United States named: “Clinical Trial Ceftriaxone in Subjects With ALS”. (ClinicalTrials.gov Identifier: NCT00349622). The purpose of this study is to evaluate the safety and efficacy of ceftriaxone treatment in amyotrophic lateral sclerosis (ALS). Participants will be randomly assigned to receive treatment with ceftriaxone or placebo for at least 12 months. The study consists of three stages. The initial phase of the trial has three arms: 1) Placebo 2×/day 2) 2 gram Ceft. 2×/day and 3) 2 gram Ceft. 1×+Placebo 1×/day. The second stage will look at the safety and side effects of the study drug when taken daily for 16 weeks. The trial was updated in Feb. 19, 2009. The duration of the second stage was extended for at least 20 weeks without the publication of the dosages of Ceftriaxone. The third stage, which began enrollment in Spring 2009, will determine whether the study drug prolong survival and slows decline in function due to ALS. According to the observations mentioned above, the whole amount of ceftriaxone applied during the treatment must be above 250 g, even in the case of very early treatment. The effective infusion treatment must start within hours or days at the latest after the appearance of the first severe complaints. Delay in the ceftriaxone administration decreases the success of treatment greatly. ALS and schizophrenia have some common features as e.g. rigidity of the muscles of the face. In the future, well planned prospective studies are needed to examine the familial coincidence of ALS and schizophrenia including morbidity and mortality from suicide among adolescent members of the involved families. The proper ceftriaxone treatment might be expanded for schizophrenic patients.

The suggested dosage for schizophrenic patients is also 2 grams, twice a day and the whole amount of ceftriaxone must be above 300 g (in cases of not very early treatment above 500 g) because, according to the observations described above, the necessary amount of ceftriaxone to provide neuroprotective effect is above 300 g, even for the mildest cases. This amount is three hundred times bigger than the effective amount of ceftriaxone to treat and cure a gonorrhoeal infection. The burden of the large amount of ceftriaxone could be decreased by administration of ceftriaxone in combinations according to the invention. (The decreased amounts of the acting agents potenciate the effectiveness of each other.) Prospective study could clarify the possibility of combined administration of decreased amount of ceftriaxone having the same or increased effectiveness for ALS and other diseases of the nervous system as the monotherapy by ceftriaxone. The locally applied present compositions can also optionally contain borax (NA₂B₄O₇.4H₂O).

The pharmaceutical compositions mentioned above can be prepared by known techniques used in the preparation of the pharmaceutical compositions.

What is claimed is:

1. A method for treating amyotrophic lateral sclerosis, comprising administering at least 100 mg/kg bodyweight/day of a treatment composition, wherein said treatment composition includes an anti-bacterial agent.

2. A method as in claim 1 wherein said anti-bacterial agent is ceftriaxone

3. A method as in claim 1, including administering said treatment composition for a period of at least eight weeks.

4. A method as in claim 1, wherein said treatment composition further comprises one or more antifungal agents effective against a Candida species, and a nitroimidazole.

5. A method as in claim 1, wherein said treatment composition further comprises an antifungal agent effective against a Candida species, and a nitroimidazole and/or a sulfonamide.

6. A method as in claim 1, wherein said treatment composition further comprises an antifungal agent effective against a Candida species, and a compound having folic acid antagonist effect and a nitroimidazole or nitroimidazole and fluor substitution.

7. A neuroprotective treatment method comprising administering to a patient a first dosage of at least 100 mg/kg bodyweight/day of ceftriaxone.

8. A neuroprotective treatment method as in claim 7, including a total dosage administration of between 300 g and 600 g.

9. A neuroprotective treatment method as in claim 7, including administering to said patient about one-half of said first dosage as a maintenance dosage.
